(19) United States
(12) Patent Application Publication
(10) Pub. No.: US 2026/0017957 A1
(43) Pub. Date: Jan. 15, 2026

(54) Method of Diagnosis

(71) Applicant: UCL Business LTD, London (GB)

(72) Inventors: Maria Francesca Cordeiro, Bray Berkshire (GB); John Maddison, Crowborough Sussex (GB)

(21) Appl. No.: 19/210,951

(22) Filed: May 16, 2025

Related U.S. Application Data
(63) Continuation of application No. 17/759,170, filed on Jul. 20, 2022, now abandoned.

(30) Foreign Application Priority Data
Jan. 22, 2020 (GB) 2000926.2

(51) Int. Cl. G06V 20/69; G06V 10/82; G16H 30/40; G16H 50/20

(52) U.S. Cl. CPC G06V 20/69; G06V 10/82; G16H 30/40; G16H 50/20; G06V 2201/03

(57) ABSTRACT

The invention relates to methods for determining the stage of a disease, particularly an ocular neurodegenerative disease such as Alzheimer's, Parkinson's, Huntington's and glaucoma, comprising the steps of identifying the status of microglial cells in the retina and relating that status to disease stage. Methods for identifying cells in the eye are also provided, as are labelled markers and the use thereof.

### CROSS REFERENCE TO RELATED APPLICATIONS

This application is a continuation of U.S. patent application Ser. No. 17/759,170, filed Jul. 20, 2022, which is a U.S. National Stage entry of International Patent Application No. PCT/EP2021/051527, filed Jan. 22, 2021, which claims priority to GB Patent Application No. 2000926.2, filed Jan. 22, 2020, under 35 U.S.C. § 119 (b). These applications are hereby incorporated by reference in their entireties.

### FIELD OF THE INVENTION

The invention relates to methods of diagnosis, particularly using images of cell death and/or activation state in the eye.

### BACKGROUND OF THE INVENTION

Cell death and neuronal loss are the key pathological drivers of neurodegeneration in conditions such as Alzheimer's (AD), Parkinson's, Huntington's and glaucoma. AD is the commonest single form of dementia predicted to increase from affecting 4 to 12 million Americans over the next 20 years. Glaucoma is the major cause of irreversible blindness throughout the world, affecting 2% of people over 40. The condition has a significant morbidity due to its silent and progressive nature, often resulting in a delay in diagnosis and treatment.

Live cell imaging has been widely used to investigate neuronal dysfunction in cultured cells in vitro, which together with fluorescent multiple-labelling permits visualisation of different cell activities and distinct molecular localization patterns. The inventors have previously reported on the ability to observe retinal ganglion cell death, using a labelled apoptotic marker (WO2009/077790) and on the usefulness of monitoring that cell death in the diagnosis of certain conditions (WO2011/055121). The inventors have now surprisingly found that it is also possible to observe the status of other cell types in the eye, in particular, the activation status of microglia cells. Further, the inventors have found that it is possible to accurately monitor the status of cells over a period of time.

### SUMMARY OF THE INVENTION

The first aspect of the present invention provides a method of determining the stage of a disease, especially a neurodegenerative disease, said method comprising the steps of identifying the activation status of microglia cells in a subject's eye and relating the status of the cells to disease stage. The step of identifying the activation status may comprise generating an image of the microglia cells.

Microglia cells are found throughout the brain and spinal cord. The cells may be in the reactive or resting (ramified) state. Reactive microglia include activated microglia and amoeboid, that is microglia that can become activated microglia. Activated microglia have antigen presenting, cytotoxic and inflammation mediating signalling ability and are able to phagocytose foreign materials. Amoeboid microglia can also phagocytose foreign material, but have no antigen presenting activity. Ramified microglia cannot phagocytose.

The inventors have found that it is possible to differentiate between reactive and ramified microglia. Further, the inventors have found that number and/or location of amoeboid, ramified or activated microglia may be used to provide an indication of the stage of disease. The presence of activated microglia is generally associated with young and or healthy people; whereas the presence of amoeboid is associated with disease. If a lower number or percentage of activated microglia and/or higher number or percentage of amoeboid microglia, than would be expected based on the subject's age or health condition, are found, it is an indication that the subject may have a neurodegenerative disease, or is likely to develop a neurodegenerative discase.

The method may comprise the step of counting the number of activated, ramified and/or amoeboid microglia in the image generated. The method may also comprise comparing the number or percentage of activated, ramified or amoeboid microglia cells found in the image with a previously obtained image, or with the expected the number or percentage of activated, ramified or amoeboid microglia. The expected number or percentage of activated, ramified or amoeboid microglia may be the number or percentage of microglia predicted based on a previous image from the same subject, or the average number or percentage of those microglia found in a similar subject of a similar age or number of subjects.

The inventors have also identified that it is possible to connect the pattern of activated, ramified and/or amoeboid microglia with disease state and with particular diseases. For example, the inventors have found that healthy subjects have an ordered and regular spread of activated microglia across the retina, but that subjects with neurodegenerative disease are more likely to have a diffuse, irregular pattern of activated microglia across the retina, or to have areas with large numbers of amoeboid microglia. The inventors have found that in glaucoma, phagocytotic microglia are generally found around the papillomacular bundle, whereas in AMD, they are found around the macula. Accordingly, the pattern of found in the image, or a change in pattern can be indicative of the subject having a neurodegenerative disease or of that disease worsening or improving. The method may comprise the step of identifying a pattern of cell status in the eye and relating that pattern to disease state.

The status of the microglia in the eye may be identified by administering a marker, particularly a labelled marker to the subject. Accordingly, the subject may be a subject to whom a labelled marker has been administrated. Alternatively, the method may also comprise administering the labelled marker to the subject. The marker may be administered in any appropriate way, particularly via intravenous injection, topically or via a nasal spray.

The inventors have surprisingly found that the labelled marker may be an apoptotic marker. The term “apoptotic marker” refers to a marker that allows cells undergoing apoptosis to be distinguished from live cells and, preferably, from necrotic cells. Apoptotic markers include, for example the annexin family of proteins. Annexins are proteins that bind reversibly to cellular membranes in the presence of cations. Annexins useful in the invention may be natural or may be recombinant. The protein may be whole or maybe a functional fragment, that is to say a fragment or portion of an annexin that binds specifically to the same molecules as the whole protein. Also, functional derivatives of such proteins may be used. A variety of annexins are available, such as those described in US Patent Application Publication No. 2006/0134001 A. A preferred annexin is annexin 5, which is well known in the art. Other annexins that may be used as apoptotic markers include annexins 1, 2 and 6. Other apoptotic markers are known in the art including for example C2A domain of synaptotagmin-I, duramycin, non-peptide based isatin sulfonamide analogs, such as WC-II-89, and ApoSense, such as NST-732, DDC and ML-10 (Saint-Hubert el ai, 2009).

The apoptotic marker is labelled, preferably with a visible label. In particular, the label is preferably a wavelength-optimised label. The term ‘wavelength-optimised label’ refers to a fluorescent substance, that is a substance that emits light in response to excitation, and which has been selected for use due to increased signal-to-noise ratio and thereby improved image resolution and sensitivity while adhering to light exposure safety standard to avoid phototoxic effects. Optimised wavelengths include infrared and near-infrared wavelengths. Such labels are well known in the art and include dyes such as IRDye700, IRDye800, D-776 and D-781. Also included are fluorescent substances formed by conjugating such dyes to other molecules such as proteins and nucleic acids. It is preferred that optimised wavelengths cause little or no inflammation on administration. A preferred wavelength-optimised label is D-776, as this has been found to cause little or no inflammation in the eye, whereas other dyes can cause inflammation. Optimised dyes also preferably demonstrate a close correlation between the level of fluorescence that may be detected histologically and that which may be detected in vivo. It is particularly preferred that there is a substantial correlation, especially a 1:1 correlation between the histological and in vivo fluorescence.

In a particular embodiment, the marker is annexin 5 labelled with D-776. The annexin 5 may be wild type annexin 5, or may be a modified annexin 5. In a particular embodiment, the annexin 5 has been modified to ensure that one molecule of annexin conjugates with one molecule of label allowing for accurate counting of cells.

The labelled apoptotic marker may be prepared using standard techniques for conjugating a wavelength-optimised label to a marker compound. Such labels may be obtained from well-known sources such as Dyomics. Appropriate techniques for conjugating the label to the marker are known in the art and may be provided by the manufacturer of the label.

An advantage of using an apoptotic marker is that the method may also be used to identify or monitor apoptosis as well as microglia status. The inventors have surprisingly found that it is further possible to differentiate between apoptosing cells to which the mark has bound and microglia cells that have phagocytosed the marker. Apoptosing cells to which the marker has bound generally appear to be ring shaped, that is round with a central hole. Activated microglia appear in two forms and can be recognised by their multiple processes. Amoeboid microglia are larger when compared to activated microglia.

The step of generating an image of the cell status may comprise generating an image of apoptosing cells. The method may also comprise counting the number of apoptosing cells and/or observing the pattern of apoptosing cells. The method may also comprise comparing the number or pattern of apoptosing cells with the expected number or pattern or with the number or pattern of apoptosing cells in an image previously generated from the subject. The apoptosing cells may particularly be retinal nerve cells such as retinal ganglion cells (RGC), bipolar, amacrine, horizontal and photoreceptor cells. In one embodiment, the cells are retinal ganglion cells. Using the combination of both apoptosing retinal nerve cells and microglia activation state allows for improved diagnosis.

It is particularly preferred to be able to monitor progression of disease or efficacy of treatment provided by comparing specific cells over time. Accordingly, the method may further comprise the step of comparing the image with an image or with more than one image of the subject's eye obtained at an earlier time point. The method may comprise comparing the number or pattern of activated and/or amoeboid microglia in one image with a previous image, and/or may comprise comparing specific cells in one image with the same cells in a previous image. A change in the activation state of microglial cells between an earlier image and a later image may be indicative of disease progression. The method may also comprise comparing the number or pattern of apoptosing cells or comparing specific cells in one image with the same cells in an earlier image, again to monitor disease progression or treatment efficacy.

The change in the number or pattern of activated or amoeboid microglia, and/or apoptosing cells can give a clinician information about the progression of disease. An increase in the number of amoeboid microglia and/or apoptosing cells may indicate disease progression. Equally, as disease reaches its later stages, a fall in the number of amoeboid or apoptosing cells may be seen. The skilled clinician is able to differentiate the stages according to the number of cells seen in one image or using a comparison with one or more further images.

When comparing specific cells, it is advantageous to be able precisely overlay one image over another. The method may comprise this step, with one, two or three or more additional images.

The disease is preferably an ocular neurodegenerative disease. The term “ocular neurodegenerative diseases” is well-known to those skilled in the art and refers to diseases caused by gradual and progressive loss of ocular neurons. They include, but are not limited to glaucoma, diabetic retinopathy, AMD, Alzheimer's disease, Parkinson's disease and multiple sclerosis.

To generate an image of cells, the labelled marker is administered to the subject, by, for example, intravenous injection, by topical administration or by nasal spray. The area of the subject to be imaged, the eye, is placed within the detection field of a medical imaging device, such as an ophthalmoscope, especially a confocal scanning laser ophthalmoscope. Emission wavelengths from the labelled marker are then imaged and an image constructed so that a map of areas of cell death is provided. Generation of the image may be repeated over a period of time. It may be monitored in real time.

It is particularly useful to be able to stage or diagnose disease, as it allows a particular treatment course to be selected and, optionally, monitored. Accordingly, the method optionally includes administering to the subject a treatment for glaucoma or another neurodegenerative disease.

Glaucoma treatments are well known in the art. Examples of glaucoma treatments are provided in the detailed description. Other treatments may be appropriate and could be selected by the skilled clinician without difficulty.

The invention also provides a labelled apoptotic marker as described herein, for use in identifying microglia activation status.

The inventors have further identified improvements to the methods of identifying cells in an image of the retina. For example, the inventors have identified improvements in methods of monitoring the status of cells in images generated using an ophthalmoscope. In particular, the cells may have been labelled with a wavelength optimised labels mentioned herein. Cell types of interest include, for example, microglia and retinal ganglion cells. The method preferably comprises the steps of:
a) providing an image of a subject's retina;
b) identifying one or more spots on each image as a candidate of a labelled cell;
c) filtering selections; and, optionally,
d) normalising the results for variations in intensity.

The spots may be identified by any appropriate method. Known methods, such as those for blob detection include template matching by convolution, connected component analysis following thresholding (static or dynamic), watershed detection, Laplacian of the gaussian, generalised hough transform and spoke filter. In one embodiment, the spots are identified by template matching.

The step of filtering the selections may be made by any appropriate method, including, for example, filtering based on calculated known image metrics such as static fixed thresholds filters, decision trees, support vector machines and random forests, which may optionally be automatically calculated for example using an autoencoder; or using the whole image and automatically calculated features and filtering using deep learning methods such as Mobilenet, Vgg16, ResNet and Inception.

The method may comprise the step of providing more than one image of the subject's retina, for example, providing images taken at different time periods. The images may have been obtained milliseconds, seconds, minutes, hours, days or even weeks apart. Where more than one image is used, the method may also comprise the step of aligning the images to ensure cells seen in one image are aligned with cells seen in the other image. The inventors have found that it is vital to align the images, in order to monitor the status of individual cells over time. It is very difficult to take repeated images of the retina and have the retina remain in exactly the same orientation in each image. It is also necessary to adapt for physical differences in the location and orientation of the patient and the eye. The inventors have surprisingly found that it is possible to align images taken at different time points and to see changes to individual cells. The step of aligning the images may comprise the step of stacking them.

The method may further comprise the step of accounting for known variants that may cause false candidate identification. This means that features in the retina, or other variants may be taken into account to reduce the likelihood of false identification of labelled cells. Such variants include non-linear intensity variation, optical blur, registration blur and low light noise, as well as biological complexities such as the patterning in the choroidal vasculature, blood vessels, blur due to cataracts, etc.

Steps of the method may be carried out by any appropriate mechanism or means. For example, they may be carried out by hand, or using an automated method. Classification steps, in particular, may be carried out by automated means, using, for example an artificial neural network.

Where steps of the method are carried out by automated means, the automated means may be trained to improve results going forward. For example, the method may further comprise the step of comparing the spots identified or classified by automated means with spots identified or classified by a manual observer or other automated means and using the results to train the first automated mechanism to better identify candidates of labelled cells.

Step a) may comprise the step of imaging the subject's retina. The retina may be imaged, for example once, twice, three, four, five or more times.

The labelled cells may be microglia cells; retinal nerve cells, especially retinal ganglion cells, or both.

The invention also provides, in accordance with other aspects, a computer-implemented method of identifying the status of cells in the retina to, for example, determine the stage of a disease, the method comprising:
a) providing an image of a subject's retina;
b) identifying one or more spots on each image as a candidate of a labelled cell;
c) filtering selections; and, optionally,
d) normalising the results for variations in intensity, as defined above.

The invention further provides, in accordance with other aspects, a computer program for identifying the status of cells in the retina to, for example, determine the stage of a disease which, when executed by a processing system, causes the processing system to:
a) provide an image of a subject's retina;
b) use template mapping to identify one or more spots on each image as a candidate of a labelled cell;
c) filter selections made by template matching using an object classification filter; and, optionally,
d) normalise the results for variations in intensity.

The methods of determining the stage of a disease described herein may be implemented using computer processes operating in processing systems or processors. These methods may be extended to computer programs, particularly computer programs on or in a carrier, adapted for putting the aspects into practice. The program may be in the form of non-transitory source code, object code, a code intermediate source and object code such as in partially compiled form, or in any other non-transitory form suitable for use in the implementation of processes described herein. The carrier may be any entity or device capable of carrying the program. For example, the carrier may comprise a storage medium, such as a solid-state drive (SSD) or other semiconductor-based RAM; a ROM, for example a CD ROM or a semiconductor ROM; a magnetic recording medium, for example a floppy disk or hard disk; optical memory devices in general; etc.

In accordance with an embodiment, there is provided a non-transitory computer-readable storage medium comprising a set of computer-readable instructions stored thereon, which, when executed by a processing system, cause the processing system to perform a method of determining the stage of a disease, the method comprising using template mapping to identify one or more spots on one or more images of a subject's retina as a candidate of a labelled cell, filtering selections made by template matching using an object classification filter, and normalising the results for variations in intensity. The described examples may be implemented at least in part by computer software stored in (non-transitory) memory and executable by the processor, or by hardware, or by a combination of tangibly stored software and hardware (and tangibly stored firmware). The method may also comprise the step of providing the one or more images of the subject's retina.

### BRIEF DESCRIPTION OF THE DRAWINGS

The invention will now be described in detail, by way of example only, with reference to the drawings.

FIG. 1 shows microglia in naïve rat and in both eyes from a glaucoma model rat (OHT and IVT).

FIG. 2 shows microglia in Alzheimer's 3×TG mouse model: aged and IVT.

FIG. 3 shows DARC & Alzheimer's 3×TG mouse model: middle-aged and IVT.

FIG. 4 shows microglia staining with Annexin V.

FIG. 5 shows the results of DARC & Alzheimer's 3×TG mouse model: nasal DARC.

FIG. 6 is a Consort Diagram Showing Glaucoma and Control Cohorts Subjects and DARC Image Analysis

FIG. 7 is a CNN-aided Algorithm Flowchart showing Analysis Stages of DARC Images

FIG. 8 is a Representative Retinal Image of the Possible Spot Candidates. Candidate spots were detected using template matching and a correlation map. Local maxima were selected and filtered with thresholds for the correlation coefficient and intensity standard deviation (corresponding to the brightness of the spot). These thresholds were set very low and produce many more spot candidates than manually observed spots (approximately 50-1).

FIG. 9 shows the CNN Training and Validation Stages CNN training (A) and validation (B) curves. A good accuracy is achieved in 200 epochs (training cycles) although training was left for 300 epochs to verify stability. The matching validation accuracy also shows similar accuracy without signs of over training. The accuracy was found to be 97%, with 91.1% sensitivity and 97.1% specificity.

FIG. 10 is a Representative Comparison of Manual Observer and CNN-algorithm DARC Spots. Spots found by the CNN and spots found by at least 2 manual observers shown on an original retinal image. (A) Patient 6, left eye. Progressive glaucoma (as measured by OCT global RFNL 3.5 ring) (B) Patient 31, left eye. Stable glaucoma. Green circles: manual observers only (False negative); Blue circles: CNN-aided algorithm only, (False Positive); Turquoise circle: Algorithm and manual observers agree (True Positive)

FIG. 11 shows ROC Curves of Glaucoma Progression of Manual Observer and CNN-algorithm analysis. Receiver Operating Characteristic (ROC) curves were constructed for both the CNN-aided algorithm (A) and manual observer 2-agree or more (B), to test predictive value of glaucoma progression at 18 months. The rate of progression (RoP) was calculated from the Spectralis OCT global retinal nerve fibre layer (RNFL) measurements at 3.5 mm from the optic disc at 18 months follow up of glaucoma subjects after DARC. Those patients with a significant (p<0.05) negative slope were defined as progressing compared to those without who were defined as stable. Maximal sensitivity (90.0%) and specificity (85.71%) were achieved at a DARC count of 23 with the AUC of 0.89 with the CNN algorithm as opposed to the manual observer count with maximal sensitivity (0.85%) and specificity (71.43%) at DARC count of 12, with the AUC of 0.79, showing the CNN-aided algorithm to be performing superiorly.

FIG. 12 shows CNN DARC counts significantly increased in glaucoma patients who go on to progress compared to those who are stable. (A) The CNN DARC count was significantly higher in patients progressing at 18 months (mean 26.13) compared to those who were stable (mean 9.71) using the CNN-aided algorithm (p=0.02). The DARC count was defined as the number of ANX776-positive spots seen in the retinal image at 120 minutes after baseline spot subtraction. (B) Although the trend was similar with manual observers (2 agree or more) DARC counts, there was no significant difference between those progressing at 18 months (mean 12.25) compared to stable (mean 4.38) glaucoma patients (p=0.0692). Box and whisker plots illustrating individual data points in glaucoma patients with and without significant RoP as measured by OCT global RFNL 3.5 ring are shown. Asterisks indicate level of significance by Mann Whitney test. Horizontal lines indicate medians and minimum and maximum ranges, and all individual data points indicated.

### DETAILED DESCRIPTION OF THE INVENTION

### Example 1

Labelled annexin V was prepared as described in WO2009077750A1. The labelled annexin was administered as described in Cordeiro MF, Guo L, Luong V, et al. Real-time imaging of single nerve cell apoptosis in retinal neurodegeneration. Proc Natl Acad Sci USA 2004; 101:13352-13356.

Iba-1 (Ionized calcium binding adaptor molecule 1 (Iba1) was used as a marker for microglia, using techniques known in the art.

Brn3a was used as a marker for retinal ganglion cells, using techniques known in the art.

Animal used included naïve rats, glaucoma model rats (OHT), Alzheimer's model mice, and glaucoma model mice. Such models are known in the art. Examples are described in WO2011055121A1.

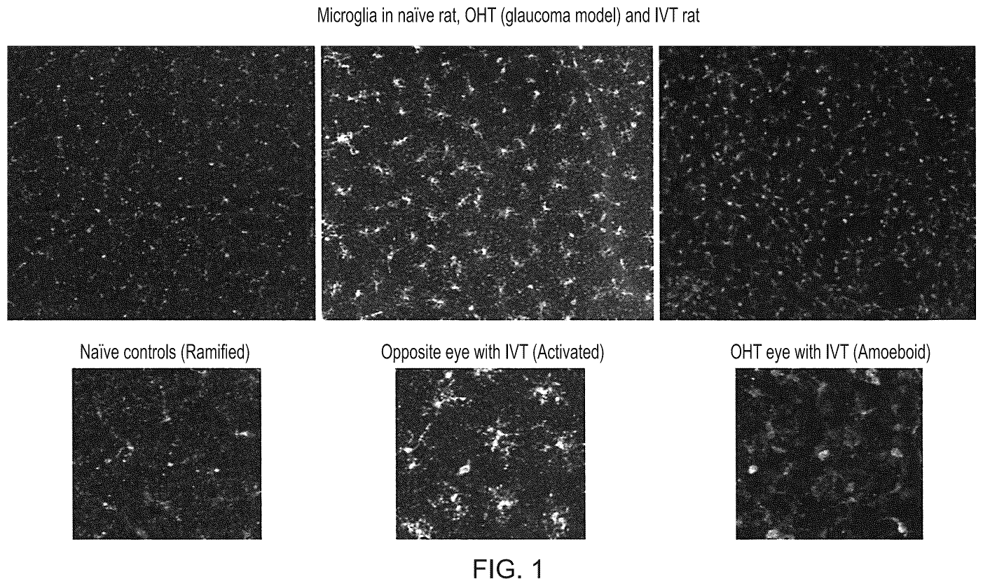

FIG. 1 shows the results of immunostaining (Iba1) of rat retinal whole mounts taken from a) naïve controls, b) opposite eyes of rats who have had surgical elevated raised IOP, (ocular hypertensive OHT model) in one eye c) the OHT eye of the same animal. Ramified, activated and amoeboid microglia can be identified.

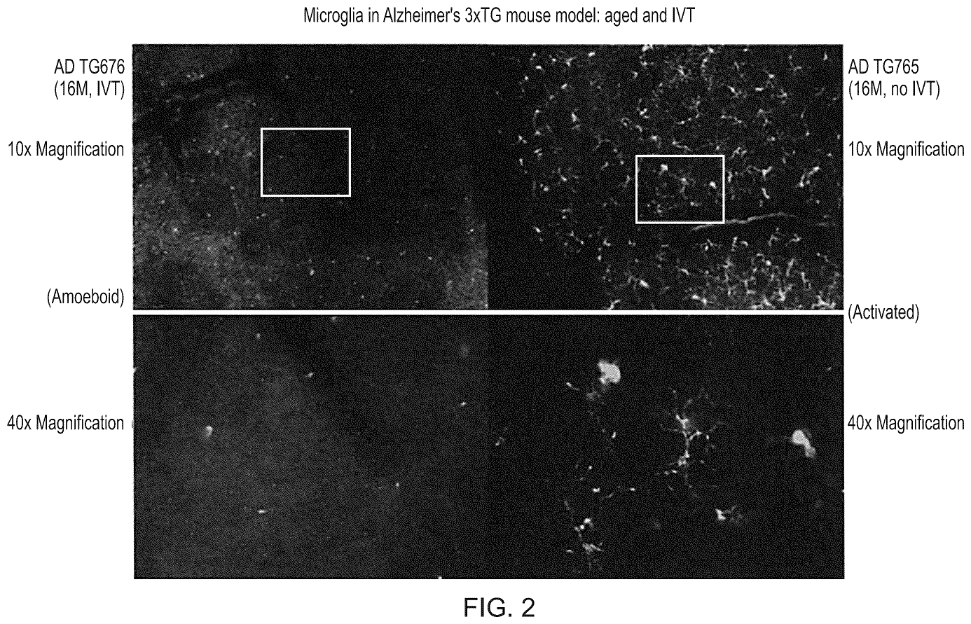

In FIG. 2, Iba1 was used to identify microglia in 16 month old Alzheimer triple transgenic mice whole retinal mounts. Following an intravitreal (IVT) injection of PBS, the morphology of the microglia contains ameoboid microglia; in contrast, and at the same age, an uninjected eye (no IVT) shows an activated morphology.

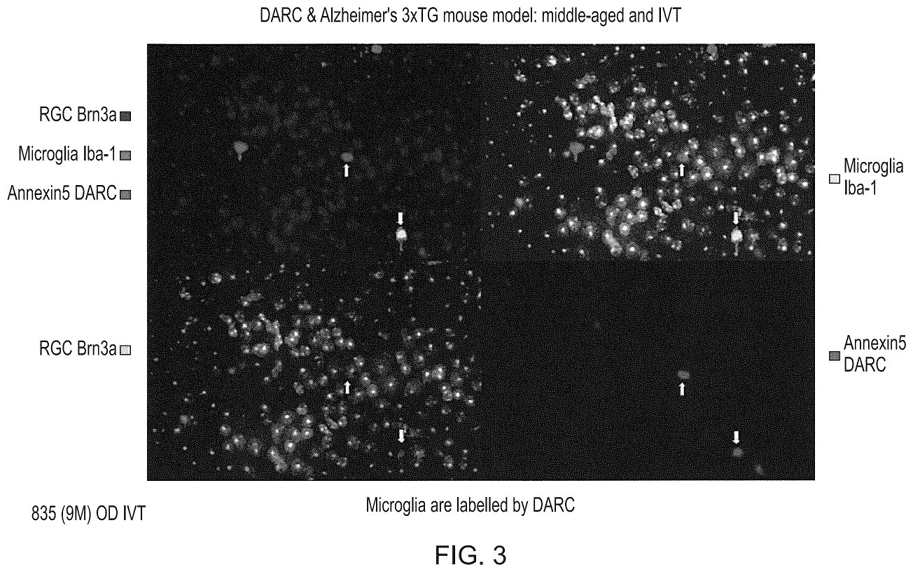

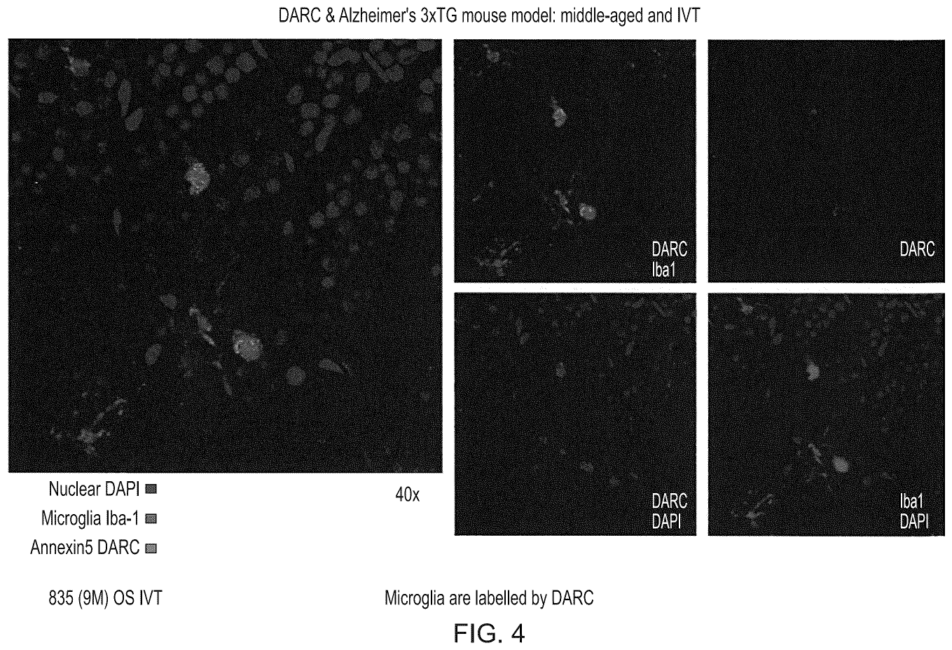

As can be seen in FIG. 3, the inventors found that it is possible to identify both retinal ganglion cells and microglia using the same stain: labelled annexin. As shown in the figure, both RGC and microglia staining can be seen, with colocalization with annexin 5. In FIG. 4, annexin 5 is fluorescently labelled with a 488 fluorescent fluorophoe which can be detected with a histological microscope. The RGC annexin staining is around the cells not within them.

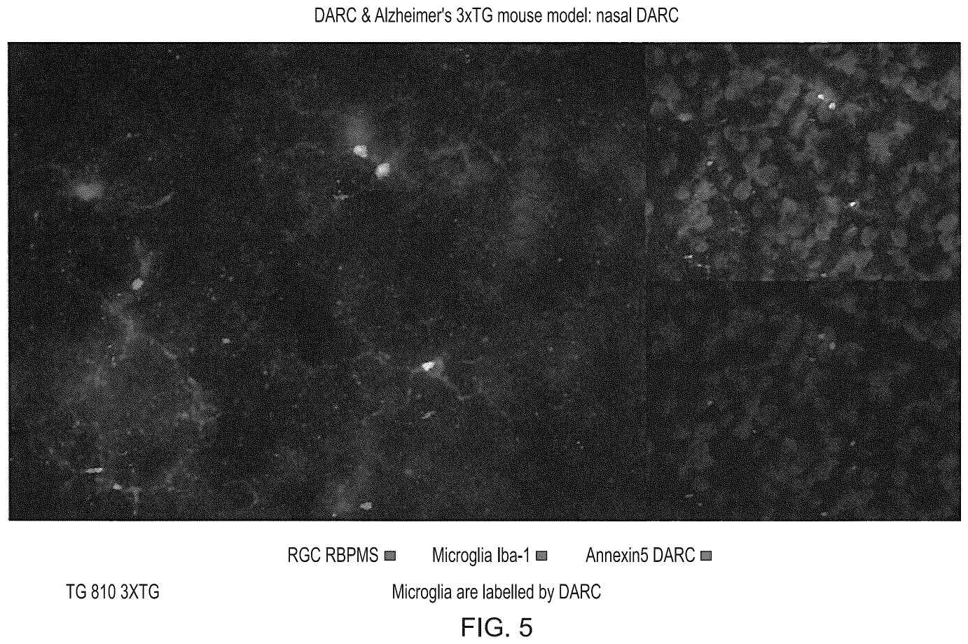

As can be seen from FIGS. 4 and 5, staining of microglia with annexin V is cytoplasmic, that is to say the annexin is intracellular or on the outside of the cell membrane as seen in RGCs.

### Example 2

Artificial intelligence is increasingly used in healthcare, especially ophthalmology. (Poplin et al., 2018) (Ting et al., 2019) Machine learning algorithms have become important analytical aids in retinal imaging, being frequently advocated in the management of diabetic retinopathy, age-related macular degeneration and glaucoma, where their utilization is believed to optimise both sensitivity and specificity in diagnosis and monitoring. (Sebastián A Banegas et al., 2015) (Quellec et al., 2017; Schmidt-Erfurth, Bogunovic, et al., 2018; Schmidt-Erfurth, Waldstein, et al., 2018; Orlando et al., 2019) The use of deep learning in these blinding conditions has been heralded as an advance to reduce their health and socio-economic impact, although their accuracy is confounded by dataset size and deficient reference standards. (Orlando et al., 2019)

Glaucoma is a progressive and slowly evolving ocular neurodegenerative disease that it is the leading cause of global irreversible blindness, affecting over 60.5 million people, predicted to double by 2040, as the aging population increases. (Quigley and Broman, 2006; Tham et al., 2014) A key objective in glaucoma research over the last few years is to identify those at risk of rapid progression and blindness. This has included, methods involving multiple levels of data including structural (optical coherence tomography (OCT), disc imaging) and functional (visual fields or standard automated perimetry (SAP)) assessments. However, several studies have demonstrated there is great variability amongst clinicians in agreement over progression using standard assessments including SAP, OCT and optic disc stereo photography. (A C Viswanathan et al., 2003) (Moreno-Montañés et al., 2017) (Sebastián A Banegas et al., 2015) However, clinical grading is regarded as the gold standard in real world practice and in deep learning datasets. (Jiang et al., 2018; Kucur, Holló and Sznitman, 2018; Asaoka et al., 2019a; Ian J C MacCormick et al., 2019; Medeiros, Jammal and Thompson, 2019a; Thompson, Jammal and Medeiros, 2019; Wang et al., 2019) Moreover, it is recognised that both OCT and SAP change only after significant death of a large number of retinal ganglion cells (RGC), (Harwerth et al., 2007) and with this the unmet need for earlier markers of disease.

Recently, we reported a novel method to visualise apoptotic retinal cells in the retina in humans called DARC (Detection of Apoptosing Retinal Cells). (Cordeiro et al., 2017) The molecular marker used in the technology is fluorescently labelled annexin A5, which has a high affinity for phosphatidylserine exposed on the surface of cells undergoing stress and in the early stages of apoptosis. The published Phase 1 results suggested that the number of DARC positively stained cells seen in a retinal fluorescent image could be used to assess glaucoma disease activity, but also correlated with future glaucoma disease progression, albeit in small patient numbers. DARC has recently been tested in more subjects in a Phase 2 clinical trial (ISRCTN10751859).

Here we describe an automatic method of DARC spot detection which was developed using a CNN, trained on a control cohort of subjects and then tested on glaucoma patients in the Phase 2 clinical trial of DARC. CNNs have shown strong performance in computer vision tasks in medicine, including medical image classification.

### Materials and Methods

### Participants

The Phase 2 clinical trial of DARC was conducted at The Western Eye Hospital, Imperial College Healthcare NHS Trust, as a single-centre, open-label study with subjects each receiving a single intravenous injection of fluorescent annexin 5 (ANX776, 0.4 mg) between 15 Feb. 2017 and 30 Jun. 2017. Both healthy and progressing glaucoma subjects were recruited to the trial, with informed consent being obtained according to the Declaration of Helsinki after the study was approved by the Brent Research Ethics Committee. (ISRCTN10751859).

All glaucoma subjects were already under the care of the glaucoma department at the Western Eye Hospital. Patients were considered for inclusion in the study if no ocular or systemic disease other than glaucoma was present and they had a minimum of three recent, sequential assessments with retinal optical coherence tomography (Spectralis SD OCT, software version 6.0.0.2; Heidelberg Engineering, Inc., Heidelberg, Germany) and standard automated perimetry (SAP, HFA 640i, Humphrey Field Analyzer; Carl Zeiss Meditec, Dublin, CA) using the Swedish interactive threshold algorithm standard 24-2. Patient eligibility was deemed possible if evidence of progressive disease in at least one eye of any parameter summarised in Table 1 & 2, was found to be present, where progression was defined by a significant (*p<0.05; **p<0.01) negative slope in the rate of progression (RoP). SAP parameters included the visual field index (VFI) and mean deviation (MD). OCT parameters included retinal nerve fibre layer (RNFL) measurements at three different diameters from the optic disc (3.5, 4.1, and 4.7 mm) and Bruch's membrane opening minimum rim width (MRW). Where it was not possible to use machine in-built software to define the rate of progression, due to the duration of the pre-intervention period of assessment, linear rates of change of each parameter with time were computed using ordinary least squares. (Wang et al., no date; Pathak, Demirel and Gardiner, 2013)

Healthy volunteers were initially recruited from people escorting patients to clinics and referrals from local optician services who acted as PICs. Healthy volunteers were also recruited from the Imperial College Healthcare NHS Trust healthy volunteers database. Potential participants were approached and given an invitation letter to participate. Participants at PICs who agreed to be contacted were approached by the research team and booked an appointment to discuss the trial. Enrolment was performed once sequential participants were considered eligible, according to the inclusion and exclusion criteria selected by the inventors. Briefly, healthy subjects were included if: there was no ocular or systemic disease, as confirmed by their GP; there was no evidence of any glaucomatous process either with optic disc, RNFL (retinal nerve fibre layer) or visual field abnormalities and with normal IOP (intraocular pressure); and they had repeatable and reliable imaging and visual fields.

### DARC Images

All participants received a single dose of 0.4 mg of ANX776 via intravenous injection following pupillary dilatation (1% tropicamide and 2.5% phenylephrine), and were assessed using a similar protocol to Phase 1. (Cordeiro et al., 2017) Briefly, retinal images were acquired using a cSLO (HRA+October Spectralis, Heidelberg Engineering GmbH, Heidelberg, Germany) with ICGA infrared fluorescence settings (diode laser 786 nm excitation; photodetector with 800-nm barrier filter) in the high resolution mode. Baseline infrared autofluorescent images were acquired prior to ANX776 administration, and then during and after ANX776 injection at 15, 120 and 240 minutes. Averaged images from sequences of 100 frames were recorded at each time point. All images were anonymised before any analysis was performed. For the development of the CNN-algorithm, only baseline and 120 minute images from control and glaucoma subjects were used.

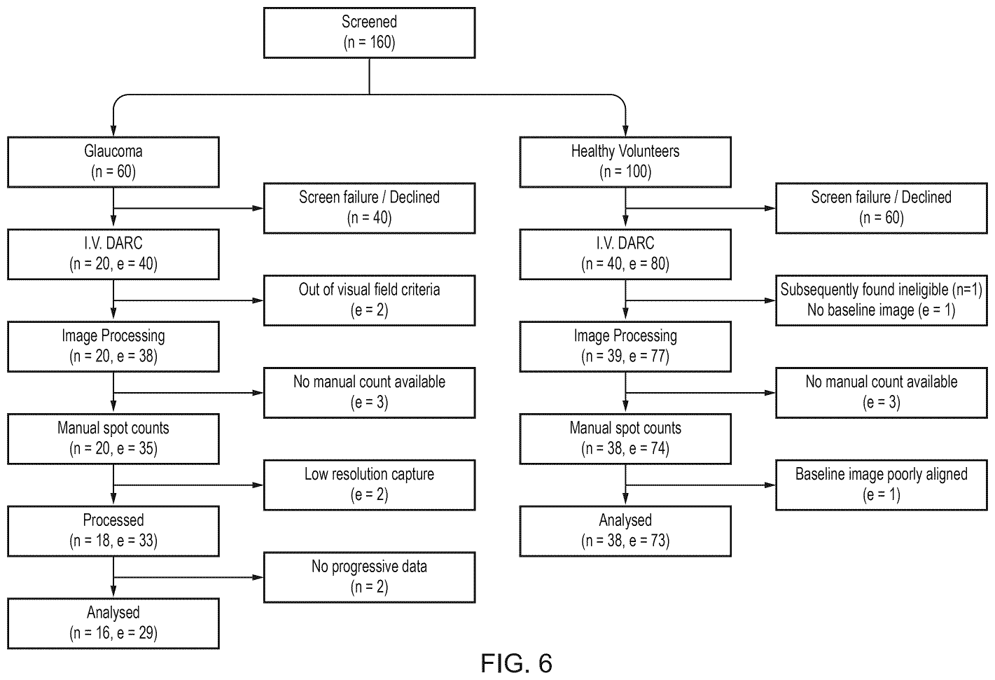

The breakdown of the images analysed are shown in the “Consort” diagram in FIG. 6. For the CNN-training, 73 control eyes at 120 minutes were available for the analysis. Similarly, of the 20 glaucoma patients who received intravenous ANX776, images were available for 27 eyes at baseline and 120 time-points.

### Manual Observer Analysis

Anonymised images were randomly displayed on the same computer and under the same lighting conditions, and manual image review was performed by five blinded operators using ImageJ® (National Institutes of Mental Health, USA). (‘ImageJ’, no date) The ImageJ ‘multi-point’ tool was used to identify each structure in the image which observers wished to label as an ANX776 positive spot. Each positive spot was identified by a vector co-ordinate. Manual observer spots for each image were compared: spots from different observers were deemed to be the same spot if they were within 30 pixels of one another. Where there was concordance of two or more observers, this was used within the automated application as the criteria for spots used to train and compare the system.

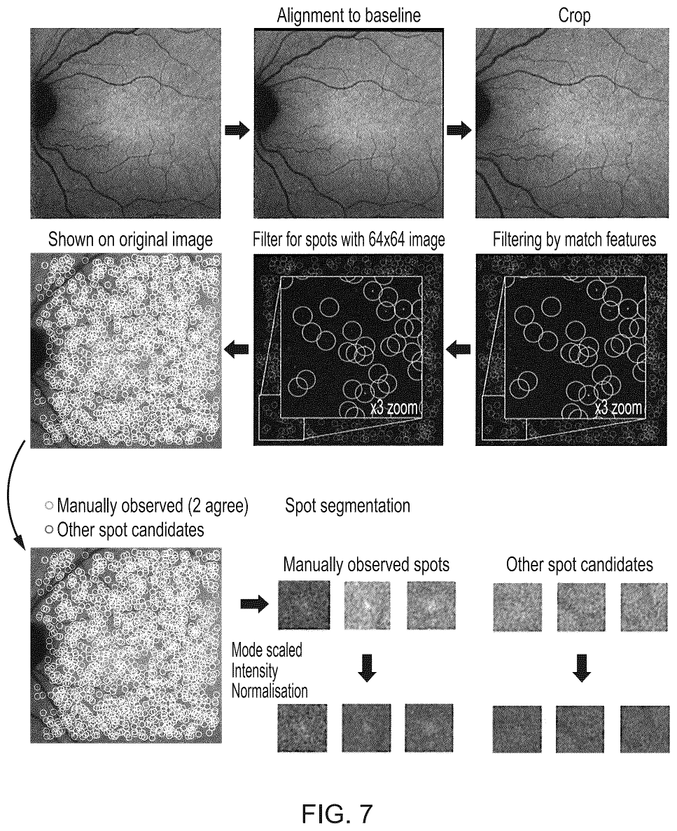

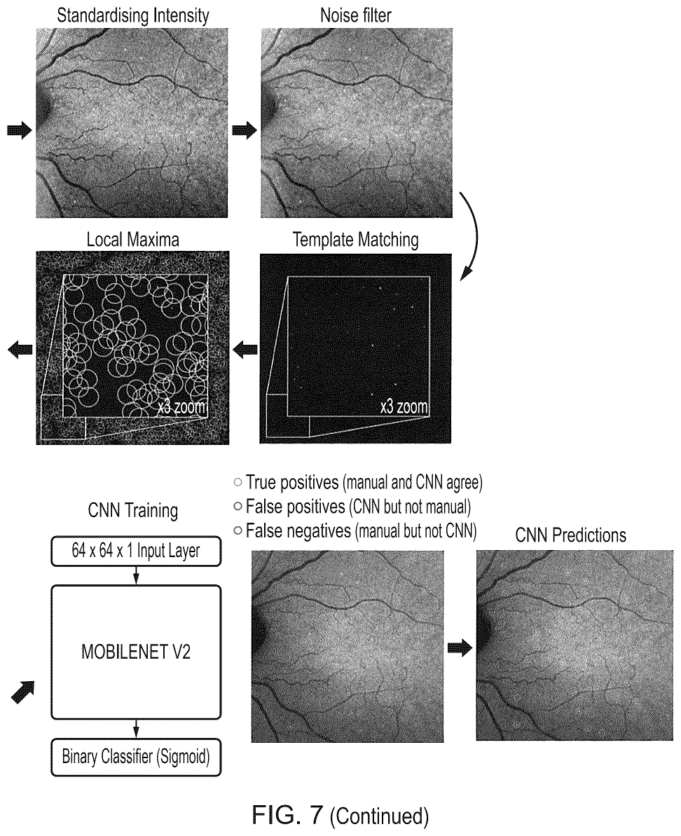

### Automated Image Analysis Overview (FIG. 7)

To detect the DARC labelled cells, candidate spots were identified in the retinal images, then classified as “DARC” or “not DARC” using an algorithm trained using the candidates and the spots identified by manual observers. FIG. 7 provides an overview of the process.

### A) Image Optimisation

Images at 120 minutes were aligned to the baseline image for each eye using an affine transformation followed by a non-rigid transformation. Images were then cropped to remove alignment artefacts. The cropped images then had their intensity standardised by Z-Scoring each image to allow for lighting differences. Finally, the high-frequency noise was removed from the images with a Gaussian blur with a sigma of 5 pixels.

### B) Spot Candidate Detection

Template matching, specifically Zero Normalised Cross-Correlation (ZNCC) is a simple method to find candidate spots. 30×30 pixel images of the spots identified by manual observers were combined using a mean image function to create a spot template. This template was applied to the retinal image producing a correlation map. Local maxima were then selected and filtered with thresholds for the correlation coefficient and intensity standard deviation (corresponding to the brightness of the spot). These thresholds were set low enough to include all spots seen by manual observers. Some of the manual observations were very subtle (arguably not spots at all) and correlation low for quite distinct spots due to their proximity to blood vessels. This means the thresholds needed to be set very low and produce many more spot candidates than manually observed spots (approximately 50-1).

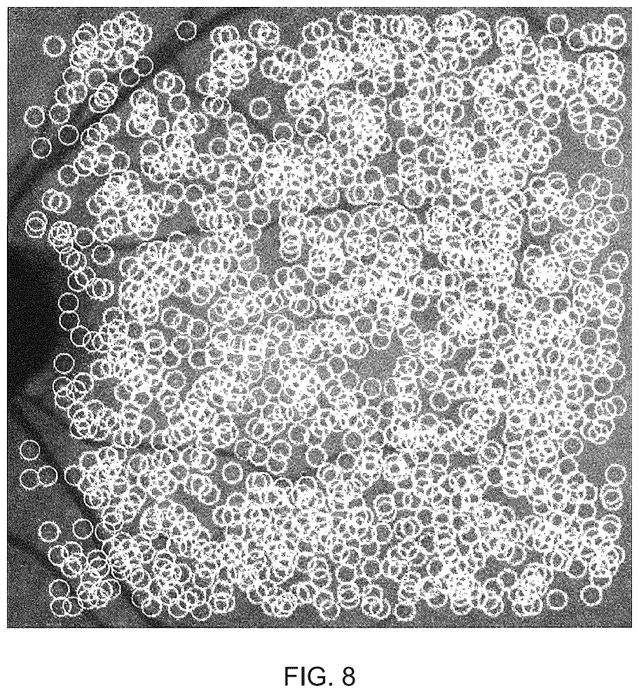

As can be seen from FIG. 8, the spot candidates cover much of the retinal image, however this reduces the number of points to classify by a factor of 1500 (compared with looking at every pixel). Using local maxima of the ZNCC, each candidate detection is centred on a spot-like object, typically with the brightest part in the centre. This means the classifier does not have to be tolerant to off-centred spots. It also means that the measured accuracy of the classifier will be more meaningful as it reflects its ability to discern DARC spots from other spot-like objects, not just its ability to discern DARC spots from random parts of the image.

### C) Spot Classification

To determine which of the spot candidates were DARC cells, the spots were classified using an established Convolutional Neural Network (CNN) called MobileNet v2. (Sandler et al., 2018; Chen et al., 2019; Pan, Agarwal and Merck, 2019; Pang et al., 2019) This CNN enables over 400 spot images to be processed in a single batch. This allows it to cope with the 50-1 unbalanced data since each batch should have about 4 DARC spots.

Although the MobileNet v2 architecture was used, the first and last layers were adapted. The first layer became a 64×64×1 input layer to take the 64×64 pixel spot candidate images (this size was chosen to include more of the area around the spot to give the network some context). The last layer was replaced with a dense layer with sigmoid activation to enable a binary classification (DARC spot or not) rather than multiple classification. An alpha value for MobileNet of 0.85 was found to work best, appropriately adjusting the number of filters in each layer

### D) Training

Training was performed only on control eyes. Briefly, retinal images were randomly selected from 120 minute images of 50% of the control patients. The CNN was trained using candidate spots, marked as DARC if 2 or more manual observers observed the spot. 58,730 spot candidates were taken from these images (including 1022 2-agree manually observed DARC spots). 70% of these spots were used to train, and 30% to validate. The retinal images of the remaining 50% of control patients were used to test the classification accuracy (48610 candidate spots of which 898 were 2-agree manually observed).

The data was augmented to increase the tolerance of the network by rotating, reflecting and varying the intensity of the spot images. The DARC spots class weights were set to 50 for spots and 1 for other objects to compensate for the 50-1 unbalanced data.

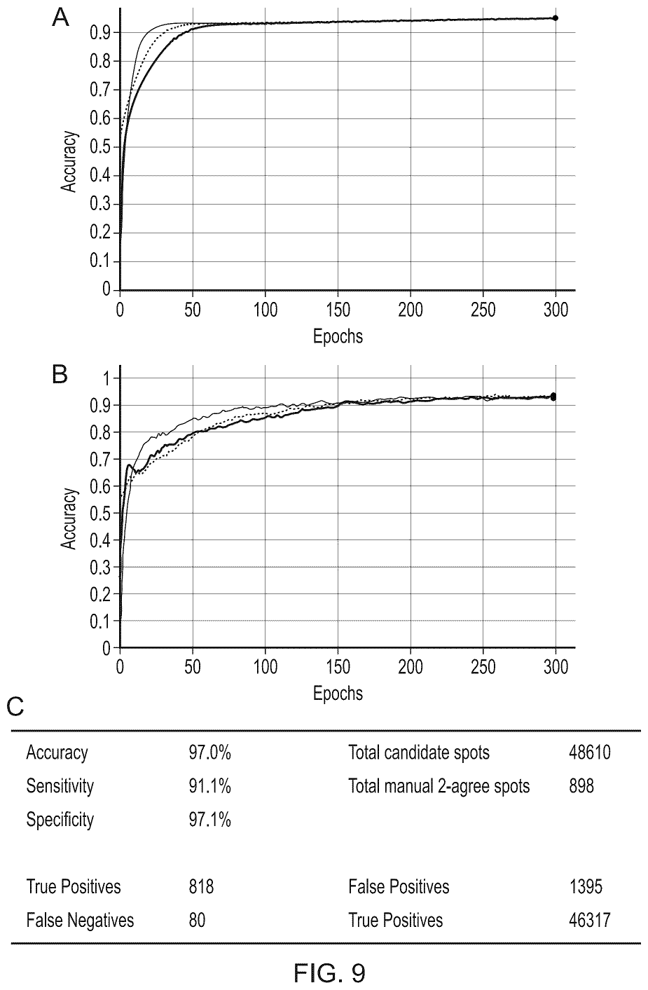

The training validation accuracy converges, and the matching validation accuracy also shows similar accuracy without signs of over training. As the training curves show (see FIG. 9) a good accuracy is achieved in 200 epochs, although training was left for 300 epochs to verify stability.

Three training runs were performed, creating three CNN models. For inference, the three models were combined: each spot was classified based on the mean probability given by each of the three models.

### E) Testing on Glaucoma DARC Images

Once the CNN-aided algorithm was developed, it was tested on the glaucoma cohort of patients in images captured at baseline and 120 minutes. Spots were identified by manual observers and the algorithm. The DARC count was defined as the number of a ANX776-positive spots seen in the retinal image at 120 minutes after baseline spot subtraction.

### Glaucoma Progression Assessment

Rates of progression were computed from serial OCTs on glaucoma patient 18 months after DARC. Those patients with a significant (p<0.05) negative slope were defined as progressing compared to those without who were defined as stable. Additionally, assessment was performed by 5 masked clinicians using visual field, OCT and optic disc measurements.

### Results

### Patient Demographics

60 glaucoma patients were screened according to set inclusion/exclusion criteria, from which 20 patients with progressing (defined by a significant (p<0.05) negative slope in any parameter in at least one eye) glaucoma underwent intravenous DARC. Baseline characteristics of these glaucoma patients are presented in Table 2. 38 eyes were eligible for inclusion, of which 3 did not have images available for manual observer counts, 2 had images captured in low resolution mode and another 2 had intense intrinsic autofluorescence. All patients apart from 2 were followed up in the Eye clinic, with data being available to perform a post hoc assessment of progression.

### Testing of Spot Classification

The results in FIG. 9 were achieved when testing the CNN-aided algorithm with the 50% of the control eyes that were reserved for test (and so were not used in training). The accuracy was found to be 97%, with 91.1% sensitivity and 97.1% specificity.

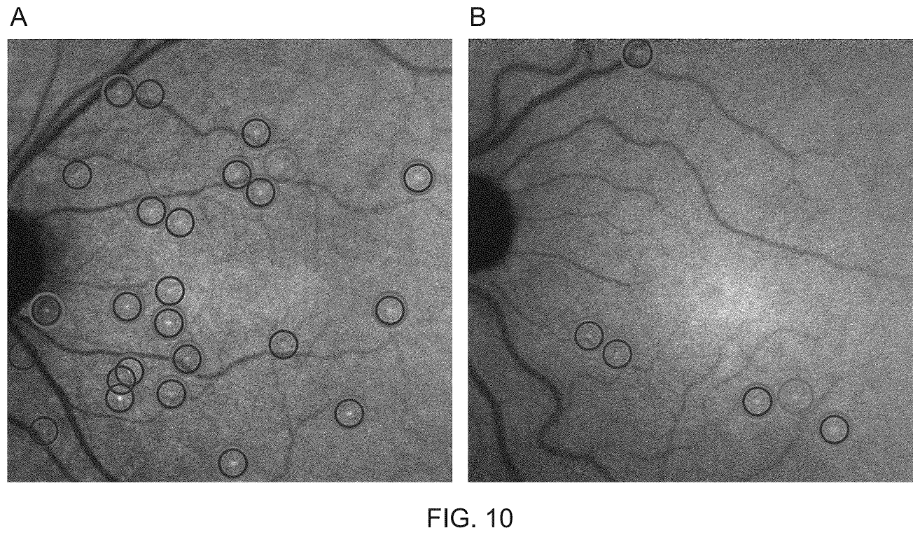

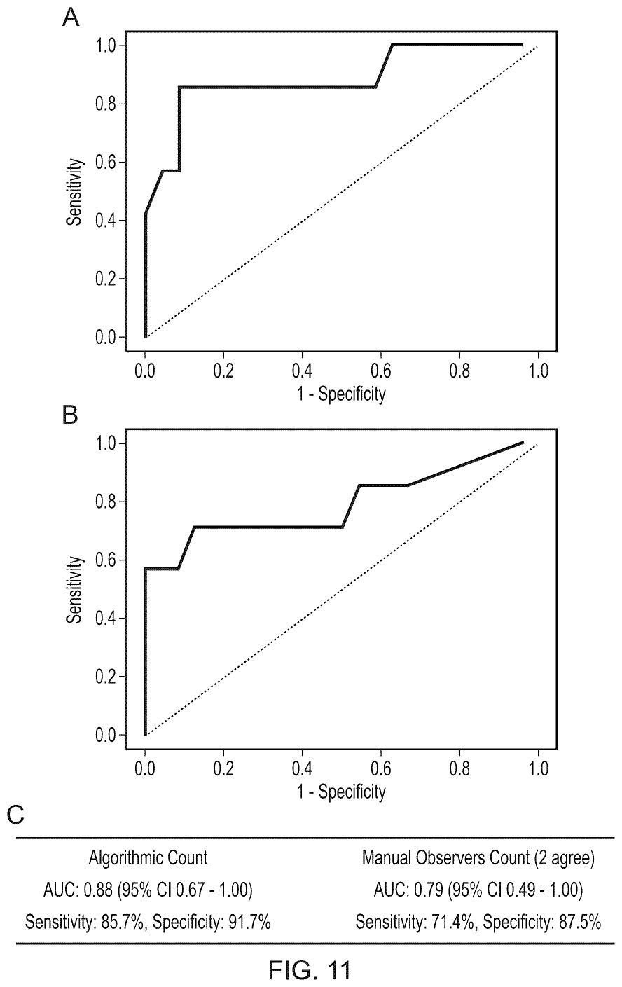

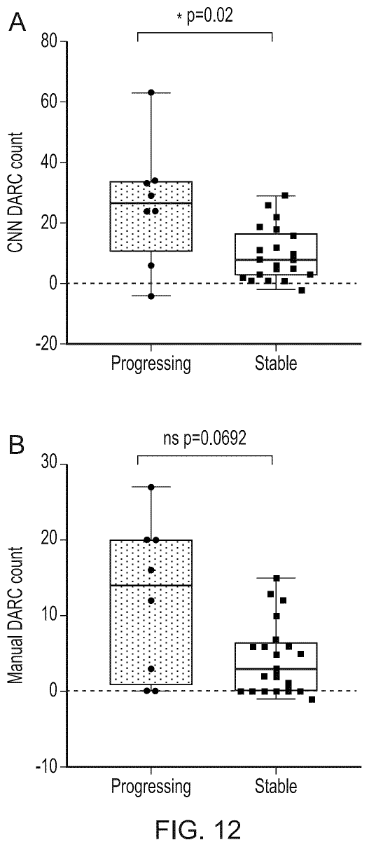

The sensitivity and specificity were encouragingly high, especially as the manual observation data that it was trained and tested on had been shown to have high levels of inter-observer variation. Typical examples of images and manual observer/algorithm spots are shown in FIG. 10.

### Classification Testing in Glaucoma Cohort

Using only the OCT global RNFL rates of progression (RoP 3.5 ring) performed at 18 months to define progression, the glaucoma cohort was divided into progressing and stable groups. Clinical agreement was poor between observers, hence, the use of objective, simple and single OCT parameter. Those patients with a significant (p<0.05) negative slope were defined as progressing compared to those without who were defined as stable, and are detailed in Table 3a. Of the 29 glaucoma eyes analysed, 8 were found to be progressing and 21 stable, by this definition.

Using this definition of glaucoma progression, a Receiver Operating Characteristic (ROC) curve was constructed for both CNN-aided algorithm and manual observer 2-agree and shown in FIG. 6, to investigate if the DARC count was predictive of glaucoma progression at 18 months. Maximal sensitivity (85.7%) and specificity (91.7%) were achieved above a

DARC count of 24 with the AUC of 0.88 with CNN algorithm as opposed to the manual observer with maximal sensitivity (71.4%) and specificity (87.5%) were achieved above a DARC count of 12, with the AUC of 0.79, showing the CNN-aided algorithm to be performing superiorly.

### DARC Counts as a Predictor of Glaucoma Progression

DARC counts in both stable and progressing glaucoma groups with the CNN-aided algorithm are shown in FIG. 7a and manual DARC counts (observer 2 agree) in FIG. 7b. The DARC count, was found to be significantly higher in patients who were later found to be progressing at 18 months (mean 26.13) compared to those who were stable (mean 9.71) using the CNN-aided algorithm (p=0.02; Mann Whitney). In comparison, manual observers (2 agree or more) DARC counts, were higher in those progressing at 18 months (mean 12.25) compared to stable (mean 4.38) glaucoma patients, but this did not reach statistical significance (p=0.0692; Mann Whitney).

### Discussion

The main goal of glaucoma management is to prevent vision loss. As the disease progresses slowly over many years, current gold standards of assessing changes not only take a long time to develop, but also after significant structural and functional damage has already occurred (Cordeiro et al., 2017). There is an unmet need in glaucoma for reliable measures to assess risk of future progression and effectiveness of treatments (Weinreb and Kaufman, 2009, 2011). Here, we describe a new CNN-aided algorithm which when combined with DARC—a marker of retinal cell apoptosis, is able to predict glaucoma progression defined by RNFL thinning on OCT, 18 months later. This method when used with DARC was able to provide an automated and objective biomarker.

The development of surrogate markers has been predominantly is cancer where they are used as predictors of clinical outcome. In glaucoma, the most common clinical outcome measure is vision loss followed by a decrease in quality of life for assessing treatment efficacy. Surrogates should enable earlier diagnoses, earlier treatment, and also shorter, and therefore more economical clinical trials. However, to be a valid surrogate marker, the measures have to be shown to be accurate. For example, OCT, which is in widespread use has been found to have a sensitivity and specificity of 83% and 88% respectively for detecting significant RNFL abnormalities (Chang et al., 2009) in addition to good repeatability (DeLeon Ortega et al., 2007) (Tan et al., 2012). In comparison, our CNN algorithm had a sensitivity of 85.7% and specificity of 91.7% to glaucoma progression.

Although the Phase 1 results suggested there was some level of DARC being predictive, this was done on a very small dataset (Cordeiro et al., 2017) with different doses of Anx776 of 0.1, 0.2, 0.4 and 0.5 mg, with a maximum of 4 glaucoma eyes per group, of which there were only 3 in the 0.4 mg group. In this present study, all subjects received 0.4 mg Anx776, and 27 eyes were analysed.

In clinical practice, glaucoma patients are assessed for risk of progression based on establishing the presence of risk factors including: older age, a raised intraocular pressure (IOP, too high for that individual), ethnicity, a positive family history for glaucoma, stage of disease, and high myopia (Jonas et al., 2017). More advanced disease risks included a vertical cup:disc ratio>0.7, pattern standard deviation of visual field per 0.2 dB increase, bilateral involvement and disc asymmetry, as also the presence of disc haemorrhages and pseudexfoliation (Gordon et al., 2002, 2003; Budenz et al., 2006; Levine et al., 2006; Miglior et al., 2007). However, none of these can be used to definitely predict individual progression.

Objective assessment is increasingly recognised as being important in glaucoma, as there is variable agreement between clinicians, even with technological aids. Poor agreement has been shown with respect to defining progression in patients using visual fields, OCT and stercophotography (A. C. Viswanathan et al., 2003) (Sebastián A. Banegas et al., 2015) (Blumberg et al., 2016) (Moreno-Montañés et al., 2017). Indeed, for this study, we asked five masked senior glaucoma specialists (co-authors) to grade for progression of patients using their clinical judgement based on optic disc assessment, OCT and visual fields; unfortunately, there was variable agreement between them (unpublished data). For this reason, a single, objective metric (Tatham and Medeiros, 2017) of rate of progression was used to define the groups used to test the CNN-aided algorithm.

The analysis of progression was post-hoc, and there was no protocol guiding treating clinicians during the 18 month period of follow-up. Similar to the oral memantine trial, (Weinreb et al., 2018) management of patients, especially with regard to IOP lowering, was left to the discretion of glaucoma specialist, and following normal standard of care. However, despite this and using the OCT global RFNL 3.5 ring RoP, 8 of 29 eyes were progressing at 18 months.

The poor agreement between clinicians identifying progression has generated great interest in the last few years in the use of artificial intelligence to help aid glaucoma diagnosis and prognosis using AI with optic disc photographs (Jiang et al., 2018) (Ian J. C. MacCormick et al., 2019) (Thompson, Jammal and Medeiros, 2019), visual fields (Pang et al., 2019) (Kucur, Holló and Sznitman, 2018) and OCT (Asaoka et al., 2019b) (Medeiros, Jammal and Thompson, 2019b). A recent study by Medeiros et al described an algorithm to assess fundus photographs based on predictions of estimated RNFL thickness, achieved by training a CNN using OCT RNFL thickness measurements (Medeiros, Jammal and Thompson, 2019b). At specificity of 95%, the predicted measurements had a sensitivity of 76% whereas actual SD OCT measurements had sensitivity of 73%. For specificity at 80%, the predicted measurements had sensitivity of 90% compared to OCT measurements which had sensitivity of 90%. The authors suggest their method could potentially could be used to extract progression information from optic disc photographs, but like our study, comment that further validation on longitudinal datasets is needed.

Template matching is routinely used for tracking cells in microscopy with similar assessment needed to analyse single cells in vivo longitudinally in this study. For template matching here, a 30×30 pixel template was used, for the CNN a 64×64 pixel image was used. The reason for this size difference is template matching is sensitive to blood vessels and so a small template is beneficial to reduce the likelihood of a blood vessel being included. For the CNN a larger image is useful to give the CNN more context of the area around the spot which may be useful in classification.

Although the algorithm performs well, providing a viable method to detect progressive glaucoma 18 months ahead of alternative methods, we believe there are areas where it can be optimised, some of which are described below.

Alternative classification algorithms to MobileNetV2 such as Support Vector Machines (SVMs) or Random Forests require “hand-crafted” features which are difficult to produce as they need to account for complexities caused by the image capture such as non-linear intensity variation, optical blur, registration blur and low light noise, as well as biological complexities such as the patterning in the choroidal vasculature, blood vessels, blur due to cataracts etc. The network has some biases to do with the intensity of the original retinal image. We believe we can improve results by looking at the intensity standardisation and augmenting the data by varying the intensity in ways more realistic with a larger dataset. The performance of other networks such as VGG16 were evaluated, at the time of writing MobiNetV2 was found to perform best. We are continuing to evaluate if this network is optimum for this need. In comparison, VGG16, an alternate CNN, would be limited to 64 spots in a batch which could mean a batch has no DARC spots in it which hinders training. We have an alternative method that detects and classifies spots in a single step using the detection and segmentation algorithm, YOLO3. We believe this may be a more efficient and effective method with more data, however at this stage the highest accuracy we have achieved with YOLO is not as good as the method outlined in this document.

### CONCLUSION

This study describes a CNN-aided algorithm to analyse DARC as a marker of retinal cell apoptosis in retinal images in glaucoma patients. The algorithm enabled a DARC count to be computed which when tested in patients was found to successfully predict OCT RNFL glaucoma progression 18 months later. This data supports use of this method to provide an automated and objective biomarker with potentially widespread clinical applications.

TABLE 1
Glaucoma Eligibility (Exclusion/Inclusion Criteria Glaucoma)

| Subject ID | Eligible eye | Diagnosis |
|---|---|---|
| 6 | Both | Primary Open Angle Glaucoma |
| 7 | Both | Glaucoma suspect |
| 9 | Both | Glaucoma suspect |
| 11 | Both | Glaucoma suspect |
| 13 | Both | Glaucoma suspect |
| 17 | Both | Glaucoma suspect |
| 18 | Both | Glaucoma suspect |
| 21 | Both | Primary Open Angle Glaucoma |
| 23 | Both | Primary Open Angle Glaucoma |
| 25 | Both | Glaucoma suspect |
| 31 | Left | Primary Open Angle Glaucoma |
| 32 | Both | Primary Open Angle Glaucoma |
| 38 | Both | Primary Open Angle Glaucoma |
| 39 | Both | Glaucoma suspect |
| 44 | Both | Primary Open Angle Glaucoma |
| 45 | Both | Glaucoma suspect |
| 52 | Both | Glaucoma suspect |
| 61 | Both | Primary Open Angle Glaucoma |
| 72 | Left | Glaucoma suspect |
| 74 | Both | Glaucoma suspect |

TABLE 1b
Glaucoma characteristics on study entry

| Diagnosis | n (%) |
|---|---|
| Glaucoma | 8 (40) |
| Glaucoma suspect | 12 (60) |
| Ocular hypertension | 0 (0) |
| | Total 20 |

TABLE 2
Baseline and Qualification Progression
parameters Glaucoma Patients

| | | OCT | | | | | |
|---|---|---|---|---|---|---|---|
| | | RNFL | RNFL | RNFL | | SAP | |
| | | 3.5 | 4.1 | 4.7 | MRW | MD | VFI |
| Subject | Eye | μm/year | μm/year | μm/year | μm/year | dB/year | %/year |
| 6 | R | | + | + | | | |
| | L | | | | | | |
| 7 | R | | | | | | |
| | L | + | | | + | | |
| 9 | R | + | | | | | |
| | L | + | | | | | |
| 11 | R | | | | | | |
| | L | + | + | | | | |
| 13 | R | | | + | + | | + |
| | L | | | + | | | |
| 17 | R | + | | | | | |
| | L | + | | | | | |
| 18 | R | + | + | | + | | |
| | L | | | | | | |
| 21 | R | + | + | | | | |
| | L | + | | | | | |
| 23 | R | | | | + | | |
| | L | + | | + | | + | |
| 25 | R | | | | | | |
| | L | + | | + | | | |
| 31 | R | + | | | | | |
| | L | | | | | | |
| 32 | R | | + | | | | |
| | L | + | | | | | |
| 38 | R | + | + | + | + | | |
| | L | + | + | + | | | |
| 39 | R | + | | + | | | |
| | L | | | | | | |
| 44 | R | + | | | | | |
| | L | + | | | | | |
| 45 | R | + | | | | | |
| | L | | | | | | |
| 52 | R | + | | | + | | |
| | L | + | | + | | | |
| 61 | R | | | | + | + | + |
| | L | + | + | + | + | | |
| 72 | R | + | | | + | | |
| | L | | + | | + | | |
| 74 | R | + | | + | | | |
| | L | + | + | + | + | | |

TABLE 3a
Progression classification per eye (OCT global
RNFL 3.5 ring) 18 months after DARC

| Category | Number of eyes |
|---|---|
| Progressing | 8 |
| Stable | 21 |
| Unknown | 4 |
| N/A | 5 |
| Total | 38 |

TABLE 3b
Clinical findings of affected eyes meeting the inclusion criteria

| | Glaucoma | | Healthy volunteer | |
|---|---|---|---|---|
| | mean (SD) | | mean (SD) | |
| BCVA, logmar | 0.01 | (0.08) | −0.03 | (0.08) |
| IOP, mmHg | 18.90 | (2.61) | 13.63 | (2.50) |
| Corneal pachimetry (CCT) | 555.58 | (33.21) | 529.99 | (25.60) |

### REFERENCES

Asaoka, R. et al. (2019a) ‘Using Deep Learning and Transfer Learning to Accurately Diagnose Early-Onset Glaucoma From Macular Optical Coherence Tomography Images’, American Journal of Ophthalmology. Elsevier Inc., 198, pp. 136-145. doi: 10.1016/j.ajo.2018.10.007.
Asaoka, R. et al. (2019b) ‘Using Deep Learning and Transfer Learning to Accurately Diagnose Early-Onset Glaucoma From Macular Optical Coherence Tomography Images’, American Journal of Ophthalmology. Elsevier Inc., 198, pp. 136-145. doi: 10.1016/j.ajo.2018.10.007.
Banegas, Sebastián A et al. (2015) ‘Agreement among spectral-domain optical coherence tomography, standard automated perimetry, and stereophotography in the detection of glaucoma progression.’, Investigative ophthalmology & visual science, 56 (2), pp. 1253-60. doi: 10.1167/iovs.14-14994.
Banegas, Sebastián A. et al. (2015) ‘Agreement among spectral-domain optical coherence tomography, standard automated perimetry, and stereophotography in the detection of glaucoma progression’, Investigative Ophthalmology and Visual Science, 56 (2), pp. 1253-1260. doi: 10.1167/iovs. 14-14994.
Blumberg, D. M. et al. (2016) ‘Technology and the glaucoma suspect’, Investigative Ophthalmology and Visual Science, 57 (9), pp. OCT80-OCT85. doi: 10.1167/iovs.15-18931.
Budenz, D. L. et al. (2006) ‘Detection and Prognostic Significance of Optic Disc Hemorrhages during the Ocular Hypertension Treatment Study’, Ophthalmology. doi: 10.1016/j.ophtha.2006.06.022.
Chang, R. T. et al. (2009) ‘Sensitivity and specificity of time-domain versus spectral-domain optical coherence tomography in diagnosing early to moderate glaucoma.’, Ophthalmology. United States, 116 (12), pp. 2294-2299. doi: 10.1016/j.ophtha.2009.06.012.
Chen, Z. et al. (2019) ‘Feature Selection May Improve Deep Neural Networks For The Bioinformatics Problems.’, Bioinformatics (Oxford, England). doi: 10.1093/bioinformatics/btz763.
Cordeiro, M. F. et al. (2017) ‘Real-time imaging of single neuronal cell apoptosis in patients with glaucoma’, Brain, 140 (6). doi: 10.1093/brain/awx088.
DeLeon Ortega, J. E. et al. (2007) ‘Effect of glaucomatous damage on repeatability of confocal scanning laser ophthalmoscope, scanning laser polarimetry, and optical coherence tomography.’, Investigative ophthalmology & visual science. United States, 48 (3), pp. 1156-1163. doi: 10.1167/iovs.06-0921.
Gordon, M. O. et al. (2002) ‘The Ocular Hypertension Treatment Study: Baseline factors that predict the onset of primary open-angle glaucoma’, Archives of Ophthalmology. doi: 10.1001/archopht.120.6.714.
Gordon, M. O. et al. (2003) ‘Ocular hypertension treatment study: Baseline factors that predict the onset of primary open-angle glaucoma’, Evidence-Based Eye Care. doi: 10.1097/00132578-200301000-00007.
Harwerth, R. S. et al. (2007) ‘The relationship between nerve fiber layer and perimetry measurements’, Invest Ophthalmol Vis Sci, 48 (2), pp. 763-773. Available at: http://www.ncbi.nlm.nih.gov/entrez/query.fcgi?cmd=Retrieve&db=PubMed&dopt=Citation&list_ui ds=17251476.
‘ImageJ’ (no date). Available at: http://imagej.nih.gov/ij/.%0A.
Jiang, Y. et al. (2018) ‘Optic Disc and Cup Segmentation with Blood Vessel Removal from Fundus Images for Glaucoma Detection’, in Proceedings of the Annual International Conference of the IEEE Engineering in Medicine and Biology Society, EMBS. Institute of Electrical and Electronics Engineers Inc., pp. 862-865. doi: 10.1109/EMBC.2018.8512400.
Jonas, J. B. et al. (2017) ‘Glaucoma’, The Lancet. doi: 10.1016/S0140-6736 (17) 31469-1.
Kucur, Ş. S., Holló, G. and Sznitman, R. (2018) ‘A deep learning approach to automatic detection of early glaucoma from visual fields.’, PloS one, 13 (11), p. e0206081. doi: 10.1371/journal.pone.0206081.
Levine, R. A. et al. (2006) ‘Asymmetries and visual field summaries as predictors of glaucoma in the ocular hypertension treatment study’, Investigative Ophthalmology and Visual Science. doi: 10.1167/iovs.05-0469.
MacCormick, Ian J C et al. (2019) ‘Accurate, fast, data efficient and interpretable glaucoma diagnosis with automated spatial analysis of the whole cup to disc profile.’, PloS one, 14 (1), p. e0209409. doi: 10.1371/journal.pone.0209409.
MacCormick, Ian J. C. et al. (2019) ‘Correction: Accurate, fast, data efficient and interpretable glaucoma diagnosis with automated spatial analysis of the whole cup to disc profile (PLoS ONE (2019) 14:1 (e0209409) DOI: 10.1371/journal.pone.0209409)’, PLoS ONE, 14 (4), pp. 1-20. doi: 10.1371/journal.pone.0215056.
Medeiros, F. A., Jammal, A. A. and Thompson, A. C. (2019a) ‘From Machine to Machine: An OCT-Trained Deep Learning Algorithm for Objective Quantification of Glaucomatous Damage in Fundus Photographs’, Ophthalmology. Elsevier Inc., 126 (4), pp. 513-521. doi: 10.1016/j.ophtha.2018.12.033.
Medeiros, F. A., Jammal, A. A. and Thompson, A. C. (2019b) ‘From Machine to Machine: An OCT-Trained Deep Learning Algorithm for Objective Quantification of Glaucomatous Damage in Fundus Photographs’, Ophthalmology. American Academy of Ophthalmology, 126 (4), pp. 513-521. doi: 10.1016/j.ophtha.2018.12.033.
Miglior, S. et al. (2007) ‘Intercurrent Factors Associated with the Development of Open-Angle Glaucoma in the European Glaucoma Prevention Study’, American Journal of Ophthalmology. doi: 10.1016/j.ajo.2007.04.040.
Moreno-Montañés, J. et al. (2017) ‘Intraobserver and interobserver agreement of structural and functional software programs for measuring glaucoma progression’, JAMA Ophthalmology. American Medical Association, 135 (4), pp. 313-319. doi: 10.1001/jamaophthalmol.2017.0017.
Orlando, J. I. et al. (2019) ‘REFUGEChallenge: A Unified Framework for Evaluating Automated Methods for Glaucoma Assessment from Fundus Photographs’, Medical Image Analysis. Elsevier BV, p. 101570. doi: 10.1016/j.media.2019.101570.
Pan, I., Agarwal, S. and Merck, D. (2019) ‘Generalizable Inter-Institutional Classification of Abnormal Chest Radiographs Using Efficient Convolutional Neural Networks.’, Journal of digital imaging, 32 (5), pp. 888-896. doi: 10.1007/s10278-019-00180-9.
Pang, S. et al. (2019) ‘An artificial intelligent diagnostic system on mobile Android terminals for cholelithiasis by lightweight convolutional neural network.’, PloS one, 14 (9), p. e0221720. doi: 10.1371/journal.pone.0221720.
Pathak, M., Demirel, S. and Gardiner, S. K. (2013) ‘Nonlinear, multilevel mixed-effects approach for modeling longitudinal standard automated perimetry data in glaucoma.’, Investigative ophthalmology & visual science, 54 (8), pp. 5505-13. doi: 10.1167/iovs.13-12236.
Poplin, R. et al. (2018) ‘Prediction of cardiovascular risk factors from retinal fundus photographs via deep learning’, Nature Biomedical Engineering. Nature Publishing Group, 2 (3), pp. 158-164. doi: 10.1038/s41551-018-0195-0.
Quellec, G. et al. (2017) ‘Deep image mining for diabetic retinopathy screening.’, Medical image analysis, 39, pp. 178-193. doi: 10.1016/j.media.2017.04.012.
Quigley, H. A. and Broman, A. T. (2006) ‘The number of people with glaucoma worldwide in 2010 and 2020’, Br J Ophthalmol, 90 (3), pp. 262-267. Available at: http://www.ncbi.nlm.nih.gov/entrez/query.fcgi?cmd=Retrieve&db=PubMed&dopt=Citation&list_ui ds=16488940.
Sandler, M. et al. (2018) ‘MobileNetV2: Inverted Residuals and Linear Bottlenecks’, arXiv e-prints The IEEE Conference on Computer Vision and Pattern Recognition (CVPR), pp. 4510-4520.
Schmidt-Erfurth, U., Bogunovic, H., et al. (2018) ‘Machine Learning to Analyze the Prognostic Value of Current Imaging Biomarkers in Neovascular Age-Related Macular Degeneration.’, Ophthalmology. Retina, 2 (1), pp. 24-30. doi: 10.1016/j.oret.2017.03.015.
Schmidt-Erfurth, U., Waldstein, S. M., et al. (2018) ‘Prediction of Individual Disease Conversion in Early AMD Using Artificial Intelligence.’, Investigative ophthalmology & visual science, 59 (8), pp. 3199-3208. doi: 10.1167/iovs.18-24106.
Tan, B. B. et al. (2012) ‘Comparison of retinal nerve fiber layer measurement between 2 spectral domain OCT instruments.’, Journal of glaucoma. United States, 21 (4), pp. 266-273. doi: 10.1097/IJG.0b013e3182071cdd.
Tatham, A. J. and Medeiros, F. A. (2017) ‘Detecting Structural Progression in Glaucoma with Optical Coherence Tomography.’, Ophthalmology. United States, 124 (12S), pp. S57-S65. doi: 10.1016/j.ophtha.2017.07.015.
Tham, Y. C. et al. (2014) ‘Global prevalence of glaucoma and projections of glaucoma burden through 2040: A systematic review and meta-analysis’, Ophthalmology. doi: 10.1016/j.ophtha.2014.05.013.
Thompson, A. C., Jammal, A. A. and Medeiros, F. A. (2019) ‘A Deep Learning Algorithm to Quantify Neuroretinal Rim Loss From Optic Disc Photographs.’, American journal of ophthalmology, 201, pp. 9-18. doi: 10.1016/j.ajo.2019.01.011.
Ting, D. S. W. et al. (2019) ‘Artificial intelligence and deep learning in ophthalmology’, British Journal of Ophthalmology. BMJ Publishing Group, pp. 167-175. doi: 10.1136/bjophthalmol-2018-313173.
Viswanathan, A C et al. (2003) Interobserver agreement on visual field progression in glaucoma: a comparison of methods, Br J Ophthalmol. Available at: www.bjophthalmol.com.
Viswanathan, A. C. et al. (2003) ‘Interobserver agreement on visual field progression in glaucoma: A comparison of methods’, British Journal of Ophthalmology, 87 (6), pp. 726-730. doi: 10.1136/bjo.87.6.726.
Wang, M. et al. (2019) ‘An Artificial Intelligence Approach to Detect Visual Field Progression in Glaucoma Based on Spatial Pattern Analysis.’, Investigative ophthalmology & visual science, 60 (1), pp. 365-375. doi: 10.1167/iovs.18-25568.
Wang, Y. X. et al. (no date) ‘Comparison of neuroretinal rim area measurements made by the Heidelberg Retina Tomograph I and the Heidelberg Retina Tomograph II.’, Journal of glaucoma, 22 (8), pp. 652-8. doi: 10.1097/IJG.0b013e318255da30.
Weinreb, R. N. et al. (2018) ‘Oral Memantine for the Treatment of Glaucoma: Design and Results of 2 Randomized, Placebo-Controlled, Phase 3 Studies’, Ophthalmology, 125 (12), pp. 1874-1885. doi: 10.1016/j.ophtha.2018.06.017.
Weinreb, R. N. and Kaufman, P. L. (2009) ‘The glaucoma research community and FDA look to the future: a report from the NEI/FDA CDER Glaucoma Clinical Trial Design and Endpoints Symposium’, Invest Ophthalmol Vis Sci, 50 (4), pp. 1497-1505. Available at: http://www.ncbi.nlm.nih.gov/entrez/query.fcgi?cmd=Retrieve&db=PubMed&dopt=Citation&list_ui ds=19321793.
Weinreb, R. N. and Kaufman, P. L. (2011) ‘Glaucoma research community and FDA look to the future, II: NEI/FDA Glaucoma Clinical Trial Design and Endpoints Symposium: measures of structural change and visual function.’, Investigative ophthalmology & visual science. United States, pp. 7842-7851. doi: 10.1167/iovs.11-7895.

1. A method of determining the stage of a disease, especially a neurodegenerative disease, comprising the steps of: a) generating an image of the activation status of microglia cells in a subject's eye and b) relating the status of the cells to disease stage.

2. The method of claim 1, further comprising one or both of steps: c) counting the number of activated, ramified and/or amoeboid microglia in the image generated; and d) comparing the number or percentage of activated, ramified or amoeboid microglia cells found in the image with a previously obtained image, or with the expected the number or percentage of activated, ramified or amoeboid microglia.

3. The method according to claim 1, further comprising the step of identifying a pattern of cell status in the eye and relating that pattern to disease state.

4. The method according to claim 1, wherein the subject is a subject to whom a labelled marker has been administrated.

5. The method according to claim 1, further comprising the step of administering a labelled marker to the subject.

6. The method of claim 4, wherein the labelled marker is an apoptotic marker, particularly a labelled annexin.

7. The method of claim 4, wherein the label is a visible label.

8. The method of claim 1, further comprising one or more of
generating an image of apoptosing cells;
counting the number of apoptosing cells and/or observing the pattern of apoptosing cells; and
comparing the number or pattern of apoptosing cells with the expected number or pattern or with the number or pattern of apoptosing cells in an image previously generated from the subject.

9. The method of claim 1, further comprising one or more of
comparing the image with an image or with more than one image of the subject's eye obtained at an earlier time point;
comparing the number or pattern of activated and/or amoeboid microglia in one image with a previous image;
comparing specific cells in one image with the same cells in a previous image; and
comparing the number or pattern of apoptosing cells or comparing specific cells in one image with the same cells in an earlier image.

10. The method of claim 9, comprising the step of overlaying one image with one, two, three or more additional images.

11. The method of claim 1, wherein the disease is an ocular neurodegenerative disease.

12. The method of claim 1, further comprising the step of determining an appropriate treatment for the subject and/or administering to the subject a treatment, particularly for glaucoma or another neurodegenerative disease.

13. A method of identifying cells in an image of the retina, comprising the steps of: a) providing an image of a subject's retina; b) identifying one or more spots on each image as a candidate of a labelled cell; c) filtering selections; and, optionally, d) normalizing the results for variations in intensity.

14. The method of claim 13, further comprising the step of providing more than one image of the subject's retina.

15. The method of claim 14, further comprising the step of aligning the images to ensure cells seen in one image are aligned with cells seen in the other image.

16. The method of claim 15, further comprising the step of accounting for known variants that may cause false candidate identification.

17. The method of claim 13, wherein at least one of the steps is carried out by an automated means, and, optionally, wherein the automated means is trained to improve results going forward.

18. The method of claim 13, wherein the labelled cells are microglia cells, retinal nerve cells, or a combination thereof.

19. The method of claim 18, wherein the labelled cells are retinal ganglion cells.
